(19) United States
(12) Patent Application Publication
(10) Pub. No.: US 2006/0083774 A1
(43) Pub. Date: Apr. 20, 2006

(54) Neurophilin ligands for treating ocular conditions

(75) Inventors: Iok-Hou Pang, Grand Prairie, TX (US); Mark R. Hellberg, Highland Village, TX (US); Abdelmoula Namil, Arlington, TX (US)

Correspondence Address: Teresa J. Schultz;Alcon Research, Ltd., Mail Code Q-148, 6201 S. Freeway, Fort Worth, TX, 76134-2099, US

(73) Assignee: Alcon, Inc.

(21) Appl. No.: 11/295,033

(22) Filed: Dec. 6, 2005

Related U.S. Application Data
(63) Continuation of application No. 10/129,724, filed on May 8, 2002, now abandoned.
(60) Provisional application No. 60/165,137, filed on Nov. 12, 1999.

(51) Int. Cl. A61K 31/4439; A61F 2/00

(52) U.S. Cl. 424/427; 514/343

(57) ABSTRACT

The use of neurophilin ligands for treating glaucoma and lowering IOP is disclosed.

This application is a continuation application of U.S. Ser. No. 10/129,724 filed May 8, 2002, which is a 371 application of PCT/US00/29320 filed Oct. 24, 2000, which claims benefit of U.S. Provisional Ser. No. 60/165,137 filed Nov. 12, 1999.

The present invention is directed to the use of neurophilin ligands for preventing or reducing the rate of visual field loss and treating the ocular hypertension associated with glaucoma.

### BACKGROUND OF THE INVENTION

The glaucomas are a heterogeneous group of optic neuropathies characterized by the cupping of the optic nerve head, thinning of the retinal nerve fiber layer, and specific changes in visual fields. Elevated intraocular pressure (IOP) is a very important risk factor for the development of the most common forms of glaucoma (Sommer A., et al., “Relationship between intraocular pressure and primary open angle glaucoma among white and black Americans,” Arch. Ophthalmol., 109:1090-1095 (1991)). Elevated IOP is believed to be caused by the increased deposition of extracellular matrix material by the trabecular meshwork cells which line the outflow pathway in the trabecular meshwork or by a decrease in the synthesis, release, and activation of matrix metalloproteinases by the trabecular meshwork cells or both. The result is that the trabecular meshwork becomes clogged and unable to perform one of its most critical functions, serving as a gateway for aqueous humor flow from the anterior chamber of the eye to the Schlemm's canal. When the aqueous humor flow out of the eye via the trabecular meshwork is diminished, IOP rises.

Due to the association between elevated IOP and glaucomatous visual field loss, glaucoma has been traditionally treated by lowering IOP medically (Sugrue, M. F, “New approaches to antiglaucoma therapy”, J. Med. Chem., 40;2793-2809(1997)), and/or by laser trabeculectomy, and/or surgically (Quigley, H. A. “Open-angle glaucoma”, New England J. Med. 328:1097-1106(1993).

Laser trabeculectomy is often effectively used to decrease elevated IOP, however, the effects of laser trabeculectomy are seldom permanent. Laser treatment of the trabecular meshwork results in a dramatic increase in cell division in a population of cells believed to serve as trabecular meshwork stem cells. This leads to a repopulation of the trabecular meshwork (Acott, T. S, et al. “Trabecular repopulation by anterior trabecular meshwork cells after laser trabeculectomy”, Am. J. of Ophthalmol, 107:1-3 (1989)). Laser treatment also induces an increase in the expression of matrix metalloproteinases in the trabecular meshwork (Parshley D. E. et al., “Laser trabeculectomy induces stromelysin expression by trabecular juxtacanalicular cells” Invest. Ophthamol. Vis. Sci. 37:795-804 (1996), Bradley, J. D. et al., “Effects of matrix metalloproteinase activity on outflow in perfused human organ culture”, Invest. Ophthalmol. Vis. Sci., 39:2649-2658 (1998)). This increase is believed to increase the rate of degradation of extracellular matrix resulting in a decrease in outflow resistance.

Elevated IOP does not always result in the occurrence of visual field loss, and visual field loss may occur at levels of IOP which are considered within the normal range. Thus, factors other than IOP may play a role in determining the occurrence of visual field loss. Degeneration of the retinal ganglion cells may be related to ischemia or a cascade of events that may have been initiated by the effects of IOP on the optic nerve that once initiated proceeds even if IOP is normalized. Various methods have been directed at treating retinal ganglion cell degeneration including the use of: polyamine antagonists (Kapin, M. A. U.S. Pat. No. 5,710,165:1998), noncompetitive inhibitors of the NMDA receptor-channel complex (Lipton, S. A., U.S. Pat. No. 5,922,773:1999), sodium channel blockers (Adorante, J. S., WO98/43612), 2-imidolin-2-yl(amino)quinoxalines (Wheeler, L. A. et al., U.S. Pat. No. 5,856,329), and EP₂ receptor agonists (Woodward, D. F. U.S. Pat. No. 5,877,211).

Immunophilins are a series of chaperone proteins which mediate the activity of immunosuppressant drugs such as FK506, rapamycin, and cyclosporin A (Pratt W. B., et al, “Steroid receptor interactions with heat shock proteins and immunophilin chaperones,” Endoc Rev, 18:306-26(1997)). Immunophilins are enriched in neurons throughout the central and peripheral nervous system, indicating that the immunophilins may play a role in neural function. The finding that FK-506 dose-dependently accelerates functional recovery from nerve injury initiated a search to determine the mechanism of this function. The finding that non-immunosuppressant analogs of FK-506 can also facilitate neuroregeneration suggested that ligands of non-immunosuppressant immunophilins (referred to herein as neurophilin ligands) may have therapeutic utility in a variety of neurodegenerative disorders such as Parkinson's disease, Alzheimer's disease, diabetic and peripheral neuropathies, and spinal cord injury (Hamilton, G. S.; Steiner, J. P., “Immunophilins: Beyond Immunosuppression,” J. Med. Chem., 41:5119-5143).

The neurotrophic properties of immunophilin ligands (such as FK-506) were believed to depend on their interaction with the 12-kDa FK506 binding protein (FKBP-12). More recent studies have suggested that the protective effects of these compounds may be mediated by interaction with the immunophilin FKBP-52 (also known as FKBP-59 or heat shock protein 56) and possibly other related immunophilins (Gold, G. G. et al., “Immunophilin FK506-binding protein 52 (not FK506-binding protein 12) mediates the neurotrophic action of FK506”, J. Pharmacol. Exp. Ther., 289:1202-1210). The use of pipecolic acid derivatives having affinity for FKBP-type immunophilins to stimulate or promote growth or regeneration including neurological disorders of the eye has been disclosed (Steiner, J. P. et al. WO 96/40140). The use of N-glyoxyl-prolyl ester compounds having an affinity for FKBP-type immunophilin for treatment of neurological disorders of the eye has also been disclosed (Hamilton, G. S., et al., WO 96/40633).

### SUMMARY OF THE INVENTION

The present invention is directed to the use of neurophilin ligands to treat glaucoma, lower and control IOP, and prevent the visual field loss associated with glaucoma in mammals.

### DETAILED DESCRIPTION OF PREFERRED EMBODIMENTS

The neurophilin ligands are a class of compounds that effectively treat glaucoma by exerting a protective effect on the retinal ganglion cells and the cells of the optic nerve head and by decreasing IOP by rejuvenating the trabecular meshwork cells. Although the exact mechanism or mechanisms which underlie the protective effect of the neurophilin ligands is not completely understood, these compounds are effective in inhibiting neuronal degeneration and in promoting neuro-regeneration in animal neurotoxicity models.

In addition to reducing the rate of retinal ganglion cell loss and thereby slowing the progressive visual field loss associated with glaucoma, the neurophilin ligands are believed to reduce elevated IOP by stimulating trabecular meshwork cell function. The neurophilin ligands have been shown to stimulate neurite outgrowth in PC12 cells, which, like the trabecular meshwork cells, are derived from the neural crest stem cells. It is believed that neurophilin ligands will stimulate both the proliferation and the activation of the trabecular meshwork cells resulting in the re-population of the trabecular meshwork and an increase in matrix metalloproteinase production or activation which results in the degradation of the extracellular debris occluding the outflow pathway.

According to both aspects, the invention preferably will be used to treat patients which have primary open angle glaucoma, chronic closed angle glaucoma, pseudoexfoliation glaucoma, normal tension glaucoma, or other sub-types of glaucoma or ocular hypertension. Administration of the drug is achieved through routes including, but not limited to, topical ocular, periocular injection, intravitreal injection, or intravitreal implant at a dose ranging from about 0.001 to about 2 mg/eye/day; systemic, including oral, transdermal, intravenous, transnasal, buccal, or subcutaneous at concentrations of about 0.01 to about 10 mg/kg/day; or using an ocular implant, such as, an intravitreal implant comprising about 0.2 to 100 mg.

The neurophilin ligands of the present invention can be used alone (including a combination of more than one neurophilin ligand) and in combination with other agents for treating glaucoma, such as, IOP lowering drugs (ex., prostaglandins, beta blockers, carbonic anhydrase inhibitors, muscarinics, sympathomimetics, alpha agonists, and serotonergics) and/or neuroprotectants (ex., calcium or sodium channel blockers, glutamate antagonists, including NMDA antagonists, anti-apoptotic agents, adenosine reuptake inhibitors, nitric oxide synthase inhibitors, vasodilators, neurotrophic factor enhancers, neurotrophic factors (such as ciliary neurotrophic factor (CNTF), and basic fibroblast growth factor (bFGF, etc.)).

The preferred neurophilin ligands are those that have neurotrophic activity, but have little or no immunosuppresant activity. For example, one skilled in the art is referred to the following patents and patent applications for their teaching of neurotrophic compounds which are lacking immunosuppressive activity: WO 99/14998 (Amgen, Method for Preventing and Treating hearing Loss using Sensorineurotrophic Compounds) and the references disclosed therein, including, a series of picecoline derivatives that act as neurophilin ligands (U.S. Pat. No. 5,696,135); a series of proline derivatives that act as neurophilin ligands (U.S. Pat. No. 5,614,547); a series of N-sulfonyl pipecolyl and prolyl derivatives that act as neurophilin ligands (U.S. Pat. No. 5,721,256); a series of heterocyclic thiesters and ketones that act as neurophilin ligands (U.S. Pat. No. 5,786,378); a series of N-glyoxyl-prolyl ester that act as neurophilin ligands (U.S. Pat. No. 5,795,908); a series of pipecolic acid derivatives that act as neurophilin ligands (U.S. Pat. No. 5,798,355); and a series of heterocyclic ester and amide derivatives that act as neurophilin ligands (U.S. Pat. No. 5,801,187).

Further compounds which can be used according to the present invention are disclosed in U.S. Pat. No. 5,840,736 (the use of a neurophilin ligand in the presence of a neurotrophic factor for stimulating neurite outgrowth); U.S. Pat. No. 5,654,332 (the use of a neurophilin ligand in the presence of a nerve growth factor for stimulating neurite outgrowth); U.S. Pat. No. 5,811,434 (the use of a neurophilin ligand in the presence of a nerve growth factor for stimulating neurite outgrowth); U.S. Pat. No. 5,780,484 (the use of a piperidine derivative as a neurophilin ligand in the presence of a nerve growth factor for stimulating neurite outgrowth); U.S. Pat. No. 5,846,979 (the use of N-oxide of heterocyclic esters, amides, thiesters, and ketones as neurophilin ligands).

Christner, C. et al. disclose a series of cycloheximide derivatives as neurophilin ligands with neuroregenerative properties (“Synthesis and Cytotoxic Evaluation of Cycloheximide Derivatives as Potemtial Inhibitors of FKBP12 with Neuroregenerative Properties”, J. Medicinal Chem. 42:3615-22, 1999) which can be used according to the methods of this invention.

One class of preferred compounds is disclosed in the above mentioned patent U.S. Pat. No. 5,840,736. Especially preferred is (S)-N-benzyl-3-(4-chlorophenyl)-2-(methyl-(2-oxo-2(3,4,5-trimethoxyphenyl)acetyl)amino)-N-(3-(pyridinyl-4-yl-)1-(2-(pyrodinyl-4-yl)-ethyl)propyl)propionamide (Timcodar).

Another class of preferred compounds is disclosed in the above mentioned patent U.S. Pat. No. 5,614,547. Especially preferred is 1-(3,3-dimethyl-1,2-dioxopentyl)-(L)-proline 3-(3-pyridinyl)propyl ester (GPI-1046).

1-6. (canceled)

7. A method for treating a mammal suffering from glaucoma, which comprises administering a pharmaceutically effective amount of at least one neurophilin ligand selected from the group consisting of picecoline derivatives that act as neurophilin ligands, N-sulfonyl pipecolyl derivatives that act as neurophilin ligands, prolyl derivatives that act as neurophilin ligands, heterocyclic thiesters that act as neurophilin ligands, heterocyclic ketones that act as neurophilin ligands, N-glyoxyl-prolyl esters that act as neurophilin ligands, pipecolic acid derivatives that act as neurophilin ligands, heterocyclic ester derivatives that act as neurophilin ligands, and heterocyclic amide derivatives that act as neurophilin ligands.

8. The method of claim 7, wherein the neurophilin ligand is (S)-N-benzyl-3-(4-chlorophenyl)-2-(methyl-(2-oxo-2-(3,4,5-trimethoxyphenyl)acetyl)amino)-N-(3-(pyridinyl-4-yl)-1-(2-(pyrodinyl-4-yl)-ethyl)propyl)propionamide.

9. The method of claim 1, wherein the neurophilin ligand is administered via a method selected from the group consisting of topical ocular, periocular injection, intravitreal injection, and intravitreal implant.

10. The method of claim 9, wherein the amount of neurophilin ligand administered is from about 0.001 to about 2 mg/eye/day.

11. The method of claim 1, further comprising administering an agent selected from the group consisting of prostaglandins, beta blockers, carbonic anhydrase inhibitors, muscarinics, sympathomimetics, alpha agonists, serotonergics, calcium channel blockers, sodium channel blockers, glutamate antagonists, anti-apoptotic agents, adenosine reuptake inhibitors, nitric oxide synthase inhibitors, vasodilators, neurotrophic factor enhancers, and neurotrophic factors.

12. A method for lowering intraocular pressure (IOP) in a mammal suffering from elevated IOP, which comprises administering a pharmaceutically effective amount of a neurophilin ligand selected from the group consisting of picecoline derivatives that act as neurophilin ligands, N-sulfonyl pipecolyl derivatives that act as neurophilin ligands, prolyl derivatives that act as neurophilin ligands, heterocyclic thiesters that act as neurophilin ligands, heterocyclic ketones that act as neurophilin ligands, N-glyoxyl-prolyl esters that act as neurophilin ligands, pipecolic acid derivatives that act as neurophilin ligands, heterocyclic ester derivatives that act as neurophilin ligands, and heterocyclic amide derivatives that act as neurophilin ligands.

13. The method of claim 12, wherein the neurophilin ligand is (S)-N-benzyl-3-(4-chlorophenyl)-2-(methyl-(2-oxo-2-(3,4,5-trimethoxyphenyl)acetyl)amino)-N-(3-(pyridinyl-4-yl)-1-(2-(pyrodinyl-4-yl)-ethyl)propyl)propionamide.

14. The method of claim 12, wherein the neurophilin ligand is administered via a method selected from the group consisting of topical ocular, periocular injection, intravitreal injection, and intravitreal implant.

15. The method of claim 14, wherein the amount of neurophilin ligand administered is from about 0.001 to about 2 mg/eye/day.

16. The method of claim 12, further comprising administering an agent selected from the group consisting of prostaglandins, beta blockers, carbonic anhydrase inhibitors, muscarinics, sympathomimetics, alpha agonists, serotonergics, calcium channel blockers, sodium channel blockers, glutamate antagonists, anti-apoptotic agents, adenosine reuptake inhibitors, nitric oxide synthase inhibitors, vasodilators, neurotrophic factor enhancers, and neurotrophic factors.

17. A method for preventing visual field loss associated with glaucoma, which comprises administering a pharmaceutically effective amount of a neurophilin ligand selected from the group consisting of picecoline derivatives that act as neurophilin ligands, N-sulfonyl pipecolyl derivatives that act as neurophilin ligands, prolyl derivatives that act as neurophilin ligands, heterocyclic thiesters that act as neurophilin ligands, heterocyclic ketones that act as neurophilin ligands, N-glyoxyl-prolyl esters that act as neurophilin ligands, pipecolic acid derivatives that act as neurophilin ligands, heterocyclic ester derivatives that act as neurophilin ligands, and heterocyclic amide derivatives that act as neurophilin ligands.

18. The method of claim 17, wherein the neurophilin ligand is (S)-N-benzyl-3-(4-chlorophenyl)-2-(methyl-(2-oxo-2-(3,4,5-trimethoxyphenyl)acetyl)amino)-N-(3-(pyridinyl-4-yl)-1-(2-(pyrodinyl-4-yl)-ethyl)propyl)propionamide.

19. The method of claim 17, wherein the neurophilin ligand is administered via a method selected from the group consisting of topical ocular, periocular injection, intravitreal injection, and intravitreal implant.

20. The method of claim 19, wherein the amount of neurophilin ligand administered is from about 0.001 to about 2 mg/eye/day.

21. The method of claim 17, further comprising administering an agent selected from the group consisting of prostaglandins, beta blockers, carbonic anhydrase inhibitors, muscarinics, sympathomimetics, alpha agonists, serotonergics, calcium channel blockers, sodium channel blockers, glutamate antagonists, anti-apoptotic agents, adenosine reuptake inhibitors, nitric oxide synthase inhibitors, vasodilators, neurotrophic factor enhancers, and neurotrophic factors.
